(19) United States
(12) Patent Application Publication
(10) Pub. No.: US 2011/0152382 A1
(43) Pub. Date: Jun. 23, 2011

(54) Composition of molecular elements for phosphorylase kinase inhibition

(76) Inventor: Madalene Choon Ying Heng, Northridge, CA (US)

(21) Appl. No.: 12/653,912

(22) Filed: Dec. 17, 2009

(51) Int. Cl. A61K 31/12; A61P 17/02; A61P 17/06; A61P 17/16

(52) U.S. Cl. 514/679

(57) ABSTRACT

Symmetrical curcuminoids molecular compounds that have been found to have anti-injury activity. It has been shown to benefit burns, injured and inflamed skin and mucous membrane and photodamaged skin. The improvement is mediated via inhibition of phosphorylase kinase activity. Phosphorylase kinase is released within 5 minutes following injury. Blocking the elevated phosphorylase kinase activity released after injury by phosphorylase kinase inhibitors, such as symmetrical curcuminoids, results in rapid reversal of burns, skin and mucous membrane injury and inflammation, repair of photodamaged skin, and prevention of scar tissue formation after injury.

### BACKGROUND OF THE INVENTION

1. Field of the Invention

The present invention relates to chemical compositions and specifically toward a molecular structure of diferuloylmethane with symmetry of elements on either side in order to provide maximal inhibition of phosphorylase kinase.

2. Description of the Prior Art

It has previously been reported that elevated phosphorylase kinase correlates with psoriatic activity, as well as increased phosphorylation events (Heng M C Y et al. Elevated phosphorylase kinase activity in psoriatic epidermis: correlation with increased phosphorylation and psoriatic activity. British Journal of Dermatology 1994; 130:297-306). Following the issue of the previous patent issued to Heng M C Y in 1999, it was also reported that curcumin-induced suppression of phosphorylase kinase activity correlated with resolution of psoriasis (Heng M C Y et al. Drug-induced suppression of phosphorylase kinase activity correlates with resolution of psoriasis as assessed by clinical, histological and immunohistochemical parameters. British Journal of Dermatology 2000; 143:937-949).

### SUMMARY OF THE INVENTION

Looking into the molecular structure of molecules with anti-phosphorylase kinase activity, the following characteristics were found: Symmetry in the molecule of the curcuminoid is essential for anti-phosphorylase kinase activity in order to achieve clinical effects (Heng unpublished data December 2009). These clinical effects include, among other things, anti-psoriatic activity. It has been found that improvement of psoriasis correlates with the suppression of phosphorylase kinase activity. Clinical improvement in psoriasis has been used to measure the effectiveness of anti-phosphorylase activity of various molecules. The greatest anti-psoriatic activity was observed with curcuminoids that are symmetrical, with far less activity in those that were asymmetrical (Heng M C Y, unpublished data 2009).

Symmetrical curcuminoids have also been found to have anti-injury activity (Heng M C Y, unpublished data 2009) Notably, it has been shown to benefit burns and photodamaged skin (Heng M C Y, unpublished data 2009). The improvement is also mediated via inhibition of phosphorylase kinase activity. Phosphorylase kinase is released within 5 minutes following injury. Blocking the elevated phosphorylase kinase activity released after injury by phosphorylase kinase inhibitors, such as symmetrical curcuminoids, results in rapid reversal of burns injury and repair of photodamaged skin (Heng M C Y, unpublished data 2009).

There is some leeway with regard to side-chain substitutions with regard to methoxy, hydrogen and hydroxyl side chains on the benzene ring. For example, the methoxy (—OCH3) group in the #2 position on the first benzene ring may be transferred to the #3, #4, or #5 positions of the benzene ring, so long that the same groups are present in a symmetrical position on the second benzene ring.

Additionally, methoxy (—OCH₃) group in #2 position on the first benzene ring may be substituted by —H or —OH groups so long that similar groups occur on the second benzene ring to form a symmetrical configuration.

Furthermore, hydroxyl (OH) group in #3 position on the first benzene ring may be substituted by (—H) or methoxy (—OCH₃) groups, so long a similar group in present on the second benzene ring to maintain a symmetrical structure. The same stipulation applies to the presence of (—OH) in positions #2, #4, or #5 position on the first benzene ring, and the maintainance of symmetry in the overall molecule.

Finally, hydrogen groups (—H) may substitute for the above side chains so long the whole molecule remains symmetrical.

### BRIEF DESCRIPTION OF THE DRAWINGS

This invention can better be understood by reference to the drawings, provided for exemplary purposes, and in which:

FIG. 1 illustrates the basic diferuloylmethane structure of curcumin molecule of the instant invention.

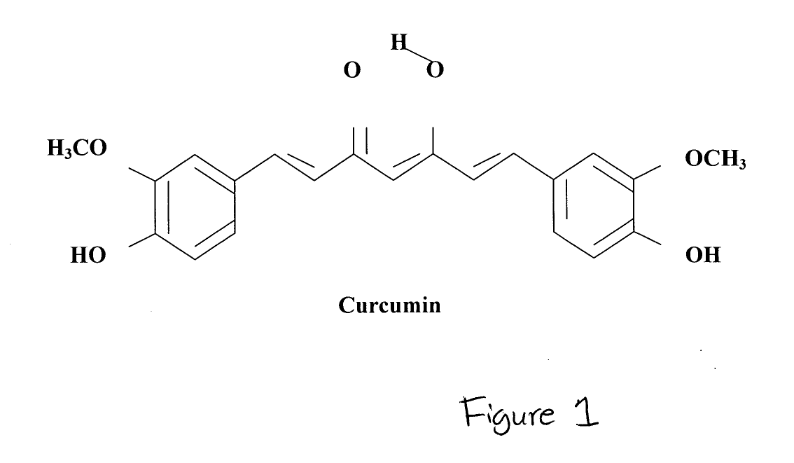

### DETAILED DESCRIPTION OF A PREFERRED EMBODIMENT

Photocarcinogenesis is caused by DNA damage from solar radiation in the ultraviolet range, resulting in the development of both melanoma and non-melanoma skin cancers. Although the UVB spectrum has previously been considered the more carcinogenic of the two, recent evidence suggests that UVA irradiation may have damaging effects that are not generally appreciated. Furthermore, it is becoming apparent that although suncreens have been in use for many years, they are relatively ineffective in protecting against UVA-induced photoaging and UVA-induced skin cancers. More recently, attention has been directed on certain dietary phytochemicals, in particular curumin, in the attempt to repair photodamaged skin as a means of preventing degeneration into solar-induced skin cancers. Curcumin has been shown to protect against the deleterious effects of injury by attenuating oxidative stress and suppressing inflammation. The ability of curcumin to block multiple targets on these pathways serve as a basis for the potential use of this phytochemical in photoaging skin and photocarcinogenesis.

Photoaging of the skin, with associated skin fragility and increased risk of both melanoma and non-melanoma skin cancers is the result of chronic damage induced by prolonged exposure to both ultraviolet B (UVB) and ultraviolet A (UVA) in natural sunlight in most individuals. Although the damaging effects of both UVB and UVA components in natural sunlight are believed to be synergistic, there is increasing evidence that the UVA, which makes up 95% of the solar ultraviolet light reaching the earth, may be the more damaging of the two. Thus, the dangers of UVA exposure in tanning salons and PUVA therapy may be underestimated and should not be ignored.

Epidemiologic studies have implicated sunlight exposure as a risk factor in the development of basal cell carcinomas and squamous cell carcinomas, although the correlation for squamous cell carcinomas to sun-exposed skin is better than for basal cell carcinomas and malignant melanomas. Point mutations and mutagenic bipyrimidine dimers have been observed with combined UVA and UVB exposure. Although point mutations of the type seen in UVB exposure have been observed in the p53 gene on chromosome 17p, such mutations have only been observed in half (40-56%) of basal cell carcinomas. Ultraviolet B which causes redness, burning and blistering, penetrates only the superficial epidermis, down to the keratinocyte layer, and is more likely to cause squamous cell carcinomas. On the other hand, the relatively asymptomatic UVA rays, with penetrating properties down to the mid and lower dermis, are probably responsible for DNA damage in basal cell carcinomas and malignant melanomas, as well as for dermal changes in photoaging. It is of interest that p53 mutations, which correlate with mutagenesis, do not correlate with photoaging.

It has been observed that sunscreens alone do not provide adequate protection against photoaging changes, nor against the development of photocarcinogenesis. The inadequacy of suncreens to protect against UVA-induced free radical formation has been reported, which probably accounts for the failure of sunscreens to prevent photoaging and photocarcinogenesis. Like x-radiation, UVA penetrates through clothing and skin, and is only blocked by bony structures, accounting for basal cell carcinomas and melanomas in areas covered by clothing. More recently, increasing interest has been focused on certain botanicals and their potential use in the treatment of photoaging skin and prevention of photocarcinogenesis. Curcumin (diferuloylmethane) is a dietary phytochemical found in the rhizome of the plant (Curcuma longa) from which turmeric is derived.

Of the two types of ultraviolet solar radiation capable of filtering through the clouds, UVB which causes redness, burning and blistering, used to be thought to be the more mutagenic of the two, while UVA, which is relatively asymptomatic, was thought to be safe. However, recent studies suggest otherwise. The well-known genotoxic effects of UVB radiation have been attributed to generation of bipyrimidine photoproducts, in particular cyclobutane pyrimidine dimers (CPDs) and [6-4]-photoproducts. However, it has been observed that UVA-induced photoproducts differ both in type and quantity from those generated by UVB. Investigators have found that, although CPDs were found with both UVB and UVA radiation, the CPDs were more abundant with UVA radiation, and were qualitatively different, resulting in less rapid removal of the UVA-induced CPD photoproducts. Furthermore, the UVA-induced CPDs were observed by ligation-mediated PCR to form predominantly at thymidine-thymidine dipyrimidines, correlating with the mutation spectrum. Ultraviolet B radiation, on the other hand, produced more [6-4]-photoproducts which were completely removed 24 hours after exposure. Quantitatively, much less conversion to Dewar photoproducts were observed with UVB than UVA-induced injury. The UVA-induced bipyrimidine photoproducts were considered the main type of DNA damage that contributed to the genotoxic effect of solar UVA radiation. These bipyrimidine products generated by UVA radiation tended to convert to Dewar photoproducts that were poorly repaired and, therefore, more mutagenic. In addition, it was observed that UVA-induced CPDs form predominantly at thymine-thymine (TT) dipyrimidines compared to thymine-cytosine (TC), cytosine-thymine (CT) or cytosine-cytosine (CC) dipyrimidines found in UVB or simulated sunlight exposure. Moreover, it was reported that there was correlation of the TT dipyrimidines with the mutation spectrum. Recently, it was also observed that UVA generates pyrimidine dimers directly in DNA, and does not require intermediary photosensitizers to transfer the energy from the UVA to DNA to produce CPDs. The TT dipyrimidine predominance in UVA-induced damage may explain the increased susceptibility of UVA radiation to induce DNA toxicity.

Further potential damaging effects of UVA-induced injury are demonstrated by observation of induction of a bystander effect in human cells treated with UVA radiation. The bystander effect is not seen with UVB radiation. The bystander effect is the induction of damage in non-irradiated cells by the presence of irradiated cells, implying involvement of mechanisms for amplification of deleterious effects in areas not exposed to the radiation. The bystander effect induced by UVA radiation as well as the ability of UVA rays to penetrate deep into the dermis, may contribute to the difficulty in blocking UVA-induced damage by sunscreens and protective clothing.

Photocarcinogenesis involves three steps: (A) tumor initiation with DNA damage induced in one or more cells as a result of the genotoxic effects of the mutagenic photoproducts, (B) tumor promotion, with clonal expansion of the clones of DNA-damaged cells, and (C) tumor transformation of the damaged clones by further DNA damage, leading to disregulated growth, and associated stromal and blood vessel changes, resulting in the acquisition of metastatic potential by damaged tissue.

It has been shown that although oxidative lesions are the main type of DNA damage involved with UVB exposure, other genotoxic products are generated with solar UVA exposure that are even more mutagenic. Unlike UVB-generated [6-4] photoproducts, which are quickly repaired, UVA exposure generated bypyrimidine photoproducts are poorly repaired and isomerize into Dewar products that are highly mutagenic. In addition, the induction of singlet oxygen formation by UVA is key to signal transcription-factor-mediated gene expression in UVA-damaged skin. The formation of thymine-thymine (TT) type cyclobutane pyrimidine dimers (CPDs) in UVA-induced damage may be associated with the greater tendency of UVA radiation to induce DNA genotoxicity. Genetic damage in the region of the promoter sequence induces the premalignant status, exhibiting proliferative properperties leading to clonal expansion.

Nuclear factor-kappa B (NF-kB) is a family of related protein dimers that bind to a common sequence on the DNA, the KB site. In the quiescent state, the NF-kB dimers are located in the cytoplasm. When activated by free radicals, radiation, endotoxin, carcinogens, ultraviolet light, tumor promoters or inflammatory cytokines, the activated NF-kB dimers, a complex made of two subunits, p50/p65, are translocated to the nucleus. NF-κB then goes on to induce transcription of over 200 genes involved in cell proliferation, cell migration, cell transformation, inhibition of apoptosis, and increased metastastatic potential.

Curcumin, the active ingredient in the spice, turmeric, is an indirect, but apparently potent inhibitor of NF-kB activation. The activation of NF-κB requires the p65 subunit to be phosphorylated at serine residues 276, 529 and 536 before it undergoes nuclear translocation. In addition, the process of activating NF-κB dimers involves the removal of the inhibitory protein IκBα by phosphorylation of its kinase (IκBα kinase).

The IkB kinases (alpha and beta) exist as a complex of two catalytic subunits (alpha and beta) with the gamma subunit (chaperone) containing a zinc finger domain required for activation of the IkB kinase. The rapid activation of IkB kinase by TNFα, a stong-inducer of NF-κB, requires both phosphorylation of serine residues (ser-171, ser-181) and receptor-mediated tyrosine residues (Tyr-188 and Tyr-199) on its beta subunit, as well as phosphorylation of the zinc finger domain on the gamma subunit. This results in rapid degradation of the inhibitory protein, IκBα. Similarly, the zinc finger domain of IκB kinase (gamma subunit) is also selectively required for signal activation by UV radiation. However, since UV light is a slow and weak inducer of NF-κB, phosphorylation occurs on ser-32 and ser-36 on IκB (beta subunit), with the phosphoacceptor sites on the activation loop serving as a recognition site for ubiquitin ligase, with resultant degradation of IκB via ubiquitin-dependent proteolysis. In both cases, degradation of the inhibitory IκBα frees the NF-κB to translocate to the nucleus, where it regulates gene transcription. In addition, phosphorylation of other serine moieties appear to affect the activity of other subunits of IκB kinase activity. For example, ser-68 phosphorylation is also involved in the activity of IκBα kinase.

Curcumin, a selective phosphorylase kinase inhibitor of phosphorylase kinase, blocks NF-kB activation as well as the activation of its IkBa kinase. It is believed that the action of curcumin is mediated through inhibition of phosphorylase kinase. Phosphorylase kinase, which is activated 5 mins after injury, including UV-induced injury, is believed to be responsible for phosphorylation of both serine/threonine and tyrosine-dependent sites on p65 subunit of NF-κB and IκB (alpha and beta) catalytic subunits. Moreover, phosphorylase kinase, which increases ATP supplies through phosphorylation of glycogen phosphorylase, is also responsible for the ATP-dependent IkB gamma chaperone protein, which maintains the zinc finger in the appropriately folded state for activation of IκBα complex. Thus, curcumin, by inhibiting phosphorylase kinase, inhibits NF-κB activation and activation and degradation of IkB by inhibiting phosphorylation of serine residues on NF-κB (p65 subunit), by inhibiting phosphorylation of serine and tyrosine residues on IκB catalytic subunits and gamma subunit, and by interfering with ATP-dependent receptor folding, ubiquitination and degradation of IkB, thereby interfering with freeing of NF-κB for nuclear translocation.

Activator protein-1 (AP-1) is a transcription activator which bears similarity to a DNA-binding protein encoded by the tumor transforming viral oncogene. The complex consists of members of the jun and fos family of proteins. The inducers of AP-1 include environmental stresses such as ultraviolet light, various growth factors, and inflammatory cytokines. AP-1 has been implicated in growth regulation and cell transformation by activating cyclin D1 gene which promotes the initiation of cells into the G1 phase of the cell cycle, and by suppressing the p53 tumor suppressor gene, which in turn leads to uncontrollable growth and cell transformation. Exposure to UV light induces activation of AP-1, the activation of which is associated with phosphorylation of both fos and jun subunits.

Curcumin suppresses the activation of AP-1 by inhibiting serine phosphorylation of the of c-jun N-terminal kinase (JNK), a serine/threonine kinase. In vitro studies on human keratinocytes, curcumin has been shown to inhibit UVB-induced gene expression by inhibiting activation of AP-1, with JNK and p38 kinase as upstream synergistic elements.

Signal transduction pathways serve as targets for chemoprevention in skin cancers. In particular, mitogen-activated protein kinase activation has been studied in UV-induced signal transduction, with p38 MAP kinase detected upstream to AP-1 activation. The MAPK pathway involves activation of MAP kinase kinase kinase (MAP3Kinase, raf-1), which then activates MAP kinase kinase (MAP2Kinase, MEKK), which in turn activates MAP kinase (MAPK). MAP kinases are growth factor-dependent receptor tyrosine kinases while the upstream kinases are serine-threonine kinases. The MAP kinases, which are responsible for activating NF-kB-induced proliferative pathways, includes extracellular signal-regulated protein kinases (ERK), c-jun N-terminal kinases (JNKs) or stress-activated protein kinases (SAPKs), and p38 kinases. Extracellular signal-regulated kinases (ERKs) are activated by growth-inducing tumor promoters, such as phorbol esters, epidermal growth factors and platelet-derived growth factor/PDGF. In skin cancers, stress activated pathways are particularly important, since stress activated promoters, such as ultraviolet light, arsenic and irradiation, which are of particular importance in skin cancer promotion, activate NF-κB through phosphorylation of JNKs, SAPKs (serine/threonine kinases) and p38 kinases (tyrosine kinases). These kinases, including JNK and p38 MAP kinases have been shown to be modulated by curcumin. Curcumin blocks phosphorylation of both serine/threonine kinases and tyrosine kinases through its inhibitory effects on phosphorylase kinase.

Growth factors are proteins that bind to receptors on the cell surface, with resultant activation of cell proliferation and/or differentiation. Growth factors that are implicated in carcinogenesis include epidermal growth factor (EGF), platelet-derived growth factor PDGF), fibroblast growth factors (FGFs), insulin-like growth factor (IGF), transforming growth factors (TGFα and TGFβ) as well as cytokine growth factors such as TNFα and IL-1. These growth factor signaling pathways are involved in non-malignant proliferation such as psoriasis as well as in proliferation of transformed cells.

Using quantitative real-time polymerase chain reaction to elucidate the effect of UVA and UVB irradiated cells with sham-irradiated cells as controls. There has been observed significant increases in mRNA levels for growth factors such as TNF-α and IL-1β, with TNF-α mRNA detected almost immediately after irradiation with both UVA and UVB, but not in sham-irradiated cells. The inhibition of curcumin-inhibited growth factor gene expression is the result both of inhibition of NF-kB activation and ERK signaling.

The binding of growth factors to its tyrosine-kinase based receptor results in phosphorylation of the receptor, activation of the receptor, and triggering of signaling pathways resulting in cell growth and proliferation. Curcumin has been shown to inhibit the tyrosine-kinase activity of this receptor, as well as to deplete the protein itself by interfering with the ATP-dependent chaperone protein which maintains the receptor in the appropriately folded state. It is probable that the effect of curcumin may be achieved through its inhibition of phosphorylase kinase. In addition to its stimulatory effect on serine/threonine kinases, phosphorylase kinase also stimulates tyrosine-kinase dependent phosphorylation, and generates ATP from breakdown of glycogen. Since inhibition of phosphorylase kinase by curcumin also depletes ATP levels, the curcumin-treated ATP-depleted cell may also have difficulty in maintaining the growth factor receptor in the appropriately folded state, resulting in inhibition of growth factor-dependent signaling.

The balance between cell survival and cell death determines the number of existing cells. In cancer, the balance is tipped towards cell survival. Cell death (apoptosis) helps to remove excess, damaged or abnormal cells. It has been observed that activation of NF-κB promotes cell survival, and down-regulation of NF-κB sensitizes the cells to apoptosis induction. Inhibition of NF-kB by curcumin promotes apoptosis of photodamaged cells, and retards development of skin malignancies, thus allowing for repair of photodamaged skin.

Apoptotic proteins include the caspase family, in particular capase 8, caspase 9, and caspase 3, which trigger DNA fragmentation when activated, leading to loss of membrane potential, and leakage of cytochrome c into the cytoplasm. Other apoptotic proteins include PARP and Bax proteins, which are also involved in the apoptotic process. It has been observed that NF-κB-dependent expression of cell survival genes block apoptosis. On the other hand, phytochemicals, such as curcumin, which inhibit NF-kB activation, sensitizes cells to apoptosis induction. Curcumin has been observed to cause p53-dependent apoptosis in human basal cell carcinoma cells, and to induce apoptosis in deregulated cyclin D1-expressed cells at the G2 phase of the cell cycle in a p53-dependent manner through activation of caspase-8, with release of cytochrome c in a mitochondrial-mediated apoptotic pathway.

Anti-apoptotic proteins such as Bcl-2 and Bcl-xL inhibit apoptosis and increase cell survival, but down-regulation of apoptosis suppressor proteins such as Bcl-2 or Bcl-xL by curcumin has been shown to induce apoptosis in cancer cell lines. This leads to activation of nuclear DNA fragmentation through mitochondrial disruption and cytochrome c release involving activation of the caspase-dependent apoptotic pathways. NF-κB-dependent expression of cell survival genes, including survivin, TRAF1 and TRAF2, block apoptosis of the photodamaged cells.¹⁹ By downregulating anti-apoptotic proteins, curcumin promotes apoptosis of photodamaged cells, thus improving photoaging skin and reduce the survival of cells that may become premalignant and malignant skin lesions.

The cell survival kinase, Akt, is a serine/threonine protein kinase activated by growth and survival factors. Akt is activated by phosphorylation at the Thr308 and Ser473. Activated Akt promotes cell survival by activating NF-kB signaling pathway, and by inhibiting apoptosis of photodamaged cells. Since the activation of NF-κB signaling is dependent on removal of its inhibitory molecule, IkBa, by IκBα kinase, inhibition of IκBα kinase would result in inhibition NF-κB. Both IkBa kinase (NF-kB activator) and Akt (survival kinase) are serine/threonine kinases, activated by phosphorylase kinase and inhibited by curcumin. Thus, curcumin promotes apoptosis of photodamaged cells both by promoting NF-kB-dependent apoptosis of photodamaged cells and by inhibiting Akt-dependent cell survival of the UV-induced DNA damaged cells. Suppression of Akt, induced by ERK ½ signaling pathways, has been reported in curcumin-induced autophagic removal of damaged cells.

Proteins that regulate the cell cycle, in particular the timing of events, are important in tumor transformation since loss of this regulation is the hallmark of the cancerous cell. These proteins are known as the cyclins, which are, in turn, regulated by cyclin-dependent kinases.

Cyclin D1, a subunit of cyclin dependent kinases, cdk-4 and cdk-6, is the rate-limiting factor regulating entry into the G1 phase of the cell cycle. Overexpression of cyclin D1 causes excessive growth promotion and dysregulation of the cell cycle associated with tumorigenesis, with increased expression related to proliferating cell nuclear antigen expression and prognosis. Curcumin blocks cell proliferation by down-regulating of cyclin D1 expression and phosphorylation events. In head and neck cancers, as well as breast, and prostate cancers, curcumin has been shown to inhibit progression of the cell cycle by downregulating the expression of cyclin D1 both at the transcriptional and post-transcriptional levels. Cyclin D1 expression is regulated by NF-κB, and suppression of NF-κB by curcumin leads to downregulation of cyclin D1. Curcumin also induces AP-1/p21-mediated G1 phase arrest of the cell cycle, retarding the proliferation of premalignant and malignant cells.

p53 is a transcription factor which functions as a tumor suppressor. It regulates many cellular processes including signal transduction and cell cycle control. It is also responsible for cellular response to DNA damage and subsequent cellular genomic stability. It activates the transcription of genes such as gene expressing p21WAF1 and Bax to induce apoptosis of DNA damaged cells, resulting in the inhibition of growth of DNA damaged cells, including cancer cells. Mutant p53 loses its ability to bind DNA effectively. Consequently, the p21WAF1 protein is not formed to regulate cell division, with resultant uncontrollable growth and tumor formation. In one study, over 90% of squamous cell carcinomas and more than 50% of basal cell carcinomas had cancers that were linked with deletion of p53 suppressor gene expression. Point mutations of the type seen in UVB exposure have been observed in the p53 gene on chromosome 17p in 40-56% of basal cell carcinomas. The antitumorigenic effect of curcumin may lie in its ability to upregulate p53 and p21WAF-1/CIP1. It has been observed that curcumin, selectively induces apoptosis in deregulated cyclin D1-expressed cycling G2 phase tumor cells in a p53-dependent manner. Curcumin also inhibits proteosomal function and induces apoptosis through mitochondrial pathways. Moreover, this phytochemical targets proliferative cells more efficiently than differentiated cells.

The penetrating properties of UVA into the dermis allow ultraviolet radiation of this wavelength band to affect dermal fibroblasts and mesenchymal tissue, inducing the production of tissue metalloproteinases. Tissue injury and resultant inflammatory response result in generation of cytokines and growth factors which activate transcription factors, such as AP-1 and NF-kB. These synergize to activate metalloproteinase promoter genes, inducing gene transcription. In the case of UVA exposure, it has been shown that singlet oxygen generated as a result of UVA exposure may mediate transcription factor-induced expression of cell adhesion molecules. The upregulation of matrix metalloproteinases, which promotes invasiveness of the tumor, and expression of cell adhesion molecules (ICAM-1), which allows tumor anchorage and vascular invasion, are intimately involved in tumor metastases.

Curcumin, has been shown to have antimetastatic effects, including antiproliferation, suppression of NF-kB activation, downregulation of antiapoptotic proteins, inhibition of both tyrosine kinase-dependent pathways (receptor-mediated MAP-kinase pathways), and serine/threonine kinase pathways (IkBa kinase, mitogen activated kinases (MAP3K and MAP2K, and Akt survival kinase). Curcumin also down-regulates the expression of matrix metalloproteinase-2 (MMP-2) and matrix metalloproteinase-9 (MMP-9). MMP-2 and MMP-9 are responsible for digestion of collagen IV in basement membranes, and collagen V in the subendothelial fibrillary component of epithelial and endothelial cells, enabling the tumor cells to invade into the dermis, as well as penetrate blood vessels. In addition curcumin has been observed to inhibit angiogenic differentiation, which functions to promote metastatic spread of tumor cells. Metalloproteinase-2 (MMP-2) expression has been shown to correlate with aggressiveness of cutaneous squamous cell carcinomas. Downregulation of these metalloproteinases by curcumin may reduce the potential for tumor invasion and metastases.

The anticarcinogenic properties of curcumin have been extensively reviewed. Curcumin appears to block carcinogenesis in a multi-targeted fashion, which may be confusing at first glance because of its complexity.

Protein kinases catalyse transfer of high energy phosphate bonds from ATP to either serine/threonine or tyrosine residues, but usually not both. This is because protein kinases, with the exception of phosphorylase kinase, allow only one configuration at its substrate binding site. In contrast, phosphorylase kinase has the ability to alter both the size and the shape of its substrate binding site. This is accomplished by the presence of a hinge joint between the subunits, which allow changes in size of the substrate binding site. In addition, the substrate binding site can be made to swivel in one plane by binding to Mn, or in another plane by binding to Mg. In this way, phosphorylase kinase is able to phosphorylase substrates of multiple specificities, including protein kinases with serine/threonine, tyrosine, phosphatidylinositol, troponin etc, as specific moieties.

It has been shown that in the phosphorylase kinase molecule, the spatial arrangement of specificity determinants can be manipulated so that phosphorylase kinase can utilize other substrates. It is possible that this flexibility may be the result of both the presence of the hinge joint between the subunits of phosphorylase kinase and the ability to alter the shape of the substrate binding site by metal ion (Mg or Mn) specificity. This flexibility enables phosphorylase kinase to take part in a multiplicity of phosphorylation reactions. The ability of phosphorylase kinase subunits to adapt to different enzyme configurations allow for phosphorylase kinase to accept many substrates, including serine/threonine kinases, tyrosine kinase and phosphatidylinositol kinase, among others.

As far as It is known, phosphorylase kinase is the only known enzyme which catalyzes the phosphorylation of glycogen phosphorylase b (inactive) to glycogen phosphorylase a (active) in glycogen phosphorylase in order to generate ATP supplies from the breakdown of glycogen. Phosphorylase kinase is a tetramer of four subunits (αβγδ)₄, with binding sites for ATP, glycogen as well as Types I and II cAMP protein kinases. The δ subunit is calmodulin. Also known as ATP-phosphorylase b phosphotransferase, phosphorylase kinase integrates multiple calcium-calmodulin dependent signaling pathways triggered by cAMP-dependent protein kinases while coupling these reactions to glycogenolysis and ATP-dependent phosphorylation. Thus, both the ATP content in the tissues and the signaling pathways activated by phosphorylase kinase may be controlled by the selective phosphorylase kinase inhibitor, curcumin. These functions are blocked by curcumin, a selective phosphorylase kinase inhibitor.

In photoaging and photocarcinogenesis, curcumin has been found to inhibit two kinds of pathways: serine/threonine kinase-dependent pathways and tyrosine kinase-dependent pathways. This includes inhibition of NF-κB dependent gene transcription, cell cycling and apoptosis by inhibiting IκBα kinase (a serine/threonine kinase); inhibition of cell proliferation by inhibition of extracellular signal-regulated MAP kinases (MEKK/MAP3K and MAP2K (both serine/threonine kinases); promoting apoptosis of photodamaged cells by inhibition of Akt cell survival kinase; and inhibiting growth and proliferation of photodamaged cells by inhibition of growth factor-dependent tyrosine kinases (a series of MAP kinases, including p38, p42 and p44 kinases). In addition, curcumin induces apoptosis in deregulated cyclin D1expressed cells at the G2 phase of the cell cycle in a p53-dependent manner. By inhibiting NF-kB activation, curcumin promotes apoptosis of photodamaged cells, thus retarding photoaging and the development of skin malignancies. By blocking signaling pathways induced by solar induced photoaging and photocarcinogenesis, curcumin is able to minimize UV-induced damage, thereby enhancing repair of photodamaged skin. In addition, by inducing apoptosis of the DNA damaged cells, curcumin protects against further damage to the DNA, in particular to the p53 suppressor gene, and promotes p53-dependent cell regulation, thus inhibiting tumor transformation.

By inhibiting phosphorylase kinase, curcumin has the unique biochemical property of blocking both the serine/threonine kinase-dependent pathways and the tyrosine kinase-dependent pathways at the same time. The importance of this effect is emphasized by the results of a recent study which demonstrated that blocking the serine/threonine-dependent pathways alone resulted in potentiation of tyrosine kinase-dependent pathways. It was observed that abrogating the serine/threonine kinase pathway by deletion of a double-stranded RNA dependent serine/threonine protein kinase (PKR), abrogated TNFα-induced serine/threonine kinases including IκBα kinase, JNK, Akt and serine/threonine MAP kinases in cell proliferation, but resulted in potentiation of tyrosine kinases such as p38 MAPK and p42/p44 MAPK. This phenomenon may explain the rebound phenomenon observed with the use of corticosteroids. Curcumin, by inhibiting phosphorylase kinase, has the ability to block both pathways simultaneously, without observation of a “rebound” when treatment is discontinued.

Unfortunately, curcumin does not seem to be well absorbed when taken orally, and high doses of curcumin have apparently failed to produce clinically or pharmacologically relevant blood levels. The curcumin molecule is metabolized to curcumin glucuronate and curcumin sulfate, and these metabolites have been shown to be absorbed into the blood stream. These metabolites, which are water soluble, do not have anti-phosphorylase kinase activity, and may be less relevant to potential cutaneous anti-photoaging and anti-carcinogenic therapy. On the other hand, they are anti-inflammatory in that they possess the ability to inhibit histamine, prostaglandins and leukotrienes, and may have relevance in suppressing inflammation in certain systemic diseases. More recently, systemic delivery of unconjugated curcumin has been made possible by packaging the curcumin encapsulated in liposomes, making it possible to treat systemic cancers such as colorectal cancer, pancreatic cancer.

The instant inventor has previously reported that topical curcumin in a gel preparation inhibits phosphorylase kinase activity in the psoriatic skin. There has also been observed complete resolution of psoriasis in over 70% of psoriatic patients within 4 weeks in a series of 647 psoriatic patients treated with topical curcumin as part of a protocol. It has previously been demonstrated that inhibition of phosphorylase kinase by curcumin gel correlates with apoptosis of cells expressing the proliferating cell nuclear antigen (PCNA) as detected by the Ki-67 immunocytochemical marker. Proliferating cell nuclear antigen (PCNA) is also expressed in both premalignant (actinic keratoses, solar lentigenes) and malignant (basal cell carcinoma, squamous cell carcinoma, malignant melanoma) as well as in non-malignant epidermal proliferation (psoriasis, eczema). In addition, it has been observed that topical curcumin (curcumin gel) used alone may also decrease scar tissue formation in 220 patients following surgery, suggesting that topical curcumin gel is capable of sufficient dermal penetration in order to modulate fibroblastic and excessive inflammatory activity within the epidermis and dermis when applied to injured skin.

It has been previously shown that inhibition of phosphorylase kinase by curcumin gel is associated with decreased T lymphocyte populations in inflammatory disease. In addition, curcumin gel has been observed to benefit many skin lesions induced by injury, including burns/scalds and surgical wounds. Curcumin gel has been observed to decrease inflammation (redness, swelling and pain), to minimize the deleterious effects of injury from burns and scalds, promoting rapid healing with minimal or no residual scarring. In post-surgical wounds, the use of extra-strength curcumin gel also allows for healing of the wounds with minimal or no scarring.

In photodamaged skin, curcumin gel applied once or twice daily has been observed to improve the texture of photodamaged skin, resulting in decreased appearance of wrinkle formation. Improvement is usually not seen before 6 months, and may take 15 months or longer. It has been observed that the more damaged the skin, the greater the improvement, with patients with minimally damaged skin showing the least improvement. It is believed that curcumin gel does not possess suncreen properties, and should be used together with a sunscreen applied over the curcumin gel when dry.

Topical curcumin has been observed to be effective in decreasing solar induced erythema in photosensitivity and rosacea, and in improving solar-induced telangiectasia. In photodamaged skin with actinic keratoses and solar lentigenes, curcumin gel has been observe to induce repair of these lesions. One of the advantages of using non-invasive therapy, such as curcumin gel, over surgical procedures in photodamaged skin is the capability of curcumin gel to repair large areas of skin, compared to limited areas improved by surgical procedures. Moreover, the curcumin-treated skin more closely resembles the appearance and texture of normal skin, without the scarring and pigmentary changes which frequently accompanies surgical procedures. Both solar lentigenes and actinic keratoses are observed to benefit from curcumin gel therapy.

As seen in treatment of over a hundred patients with photodamaged skin, it is not uncommon to find that in any one patient, multiple actinic keratoses resolve in this manner, usually within 6 months or longer, while a few continue to enlarge. It is recommended that actinic keratoses that fail to improve or resolve within 6 months be biopsied to rule out early squamous cell carcinoma. While curcumin gel may be capable of causing apoptosis in premalignant lesions such as actinic keratoses, problems with penetration may limit its use in malignant tumors. It is of interest that curcumin has been found to have potent antiproliferative and proapoptotic effects on melanoma cells.

The illustrations and examples provided herein are for explanatory purposes and are not intended to limit the scope of the appended claims. This disclosure is to be considered an exemplification of the principles of the invention and is not intended to limit the spirit and scope of the invention and/or claims of the embodiment illustrated. Those skilled in the art will make modifications to the invention for particular applications of the invention.

What is claimed is:

1. A composition for compounds having anti-phosphorylase kinase activity, including the active ingredient of curcuminoid, said curcuminoid having the basic structure of diferuloylmethane (C21H9O6), said curcuminoid terminating in a first end with a first benzene ring with a first methoxy group in the #2 position on said first benzene ring, and a hydroxyl group in the #3 position on said first benzene ring and terminating in a second end with a second benzene ring with a second methoxy group in the #2 position on said second benzene ring, and a hydroxyl group in the #3 position on said second benzene ring and wherein said molecule is substantially symmetrical for unexpected improvement in clinical applications for the treatment of psoriasis, burns, skin damage, and scar prevention.

2. The composition as defined in claim 1 wherein side-chain substitutions are made to the curcuminoid wherein the first methoxy (—OCH₃) group in the #2 position on the first benzene ring may be transferred to the #3, #4, or #5 positions of the first benzene ring wherein the same groups are present in a symmetrical position on the second benzene ring.

3. The composition as defined in claim 1 wherein side chain substitutions are made to the curcuminoid wherein the first methoxy (—OCH₃) group in #2 position on the first benzene ring may be substituted by —H or —OH groups wherein the same groups are present in a symmetrical position on the second benzene ring.

4. The composition as defined in claim 1 wherein side chain substitutions are made to the curcuminoid wherein the first hydroxyl (OH) group in #3 position on the first benzene ring may be substituted by (—H) or methoxy (—OCH3) groups wherein the same groups are present in a symmetrical position on the second benzene ring.

5. The composition as defined in claim 1 wherein side chain substitutions are made to the curcuminoid wherein (—OH) groups are placed in positions #2, #4, or #5 on the first benzene ring wherein the same groups are present in a symmetrical position on the second benzene ring.

6. The composition as defined in claim 1 wherein side chain substitutions are made to the curcuminoid wherein hydrogen groups (—H) are placed in positions #2, #4 or #5 on the first benzene ring wherein the same groups are present in a symmetrical position on the second benzene ring.

7. A method of treating psoriasis, burns and skin damage comprising the steps of:
preparation of a compositions having anti-phosphorylase kinase activity, including the active ingredient of curcuminoid, said curcuminoid having the basic structure of diferuloylmethane (C21H9O6), said curcuminoid terminating in a first end with a first benzene ring with a first methoxy group in the #2 position on said first benzene ring, and a hydroxyl group in the #3 position on said first benzene ring and terminating in a second end with a second benzene ring with a second methoxy group in the #2 position on said second benzene ring, and a hydroxyl group in the #3 position on said second benzene ring; and
applying said compound to affected skin or mucous membranes.

8. The method as defined in claim 7 wherein side-chain substitutions are made to the curcuminoid wherein the first methoxy (—OCH₃) group in the #2 position on the first benzene ring may be transferred to the #3, #4, or #5 positions of the first benzene ring wherein the same groups are present in a symmetrical position on the second benzene ring.

9. The method as defined in claim 7 wherein side chain substitutions are made to the curcuminoid wherein the first methoxy (—OCH₃) group in #2 position on the first benzene ring may be substituted by —H or —OH groups wherein the same groups are present in a symmetrical position on the second benzene ring.

10. The method as defined in claim 7 wherein side chain substitutions are made to the curcuminoid wherein the first hydroxyl (OH) group in #3 position on the first benzene ring may be substituted by (—H) or methoxy (—OCH₃) groups wherein the same groups are present in a symmetrical position on the second benzene ring.

11. The method as defined in claim 7 wherein side chain substitutions are made to the curcuminoid wherein (—OH) groups are placed in positions #2, #4, or #5 on the first benzene ring wherein the same groups are present in a symmetrical position on the second benzene ring.

12. The method as defined in claim 7 wherein side chain substitutions are made to the curcuminoid wherein hydrogen groups (—H) are placed in positions #2, #4 or #5 on the first benzene ring wherein the same groups are present in a symmetrical position on the second benzene ring.
